(19) United States
(12) Patent Application Publication
(10) Pub. No.: US 2005/0193478 A1
(43) Pub. Date: Sep. 8, 2005

(54) Goggle attachment system

(76) Inventor: Patrick P. Hussey, Carlsbad, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/042,339

(22) Filed: Jan. 24, 2005

Related U.S. Application Data
(63) Continuation-in-part of application No. 10/695,055, filed on Oct. 27, 2003.
(63) Continuation-in-part of application No. 10/763,361, filed on Jan. 23, 2004.
(60) Provisional application No. 60/538,912, filed on Jan. 23, 2004.

(51) Int. Cl. A61F 9/02

(52) U.S. Cl. 2/436

(57) ABSTRACT

A goggle (12) having removable/replaceable outriggers (16) conveniently detachably engaged with the sidewalls (14) of the goggles. The outriggers (16) have an aperture (26) in communication with a venting aperture (30) communicating through the sidewall (14) to the eye cavity (28). Pins (18) project from flexible/compressible top and bottom walls of the outrigger (16) to engage within receiving cavities (24) formed in the goggle sidewalls (14).

### CROSS-REFERENCE TO RELATED APPLICATION

This application claims the benefit of U.S. Provisional Patent Application No. 60/538,912, filed Jan. 23, 2004. This application is a continuation-in-part of U.S. patent application Ser. No. 10/695,055, filed Oct. 27, 2003, incorporated herein by reference. The present application is also a continuation-in-part of U.S. patent application Ser. No. 10/763,361, filed Jan. 23, 2004, incorporated herein by reference.

### BACKGROUND OF THE INVENTION

1. Field of the Invention

The present invention relates to engagement of protective eyewear on the head of a user. More particularly it relates to an outrigger based system of mounting for goggles which renders the elastic strap or other means of engagement of the goggle removable from the goggle. Further, the outrigger being removable allows the user to adjust the aerodynamic design of the aperture communicating through the outrigger to fit the task at hand. The rotationally engaged outrigger has an aperture communicating therethrough situated behind a formed aerodynamic surface which creates a negative air pressure region adjacent to the aperture when moving air flows over the outer surface of the outrigger. This negative air pressure region is formed no matter where in its rotational mount the outrigger is situated.

Since the outrigger is easily removably engageable to the goggle, the user may attach any of a plurality of straps or other mounting systems to the goggle and concurrently or independently adjust both the aperture size and the aerodynamic characteristics of the outrigger to meet the needs for the sport or other task of the user. Further, the aperture in the outrigger contains a plurality of fins which aid in the directing of the air exhausted from the eye cavity of the goggle upward or downward. By making the outriggers easily removable, the user can also adjust this exhaust flow to meet their needs using outriggers having fins with different directional angles.

2. Prior Art

Goggles are commonly worn by participants in many sports such as skiing, motorcycling, paintballing, field hockey, lacrosse, auto racing, and other sports which present an inherent danger to the participant's eyes and head. Goggles used in combination with helmets are frequently used in sports as well as being a requirement for the military in areas with blowing sand, blowing water, or during parachute jumps, where solders need protection from the elements for their eyes and heads.

Commonly however, the goggles are designed to be worn over the eyes, and the helmet is designed for wearing over the head, but the goggles and helmet are not designed for easy concurrent use on the head of the wearer. Further, even if the user is able to fit both the helmet and goggle on their head, frequently the two components are subject to wind interference due to the gaps and air pockets between the goggles and helmet.

Further, even when goggles are worn on the head without a helmet, different sports and/or military or industrial activities can require different airflows through the goggle eye cavity to prevent fogging. As is obvious, a parachutist traveling one hundred miles an hour downward through different temperature zones can have different airflow requirements through the eye cavity to prevent fogging than a skier who is simply traversing down a mountain at thirty miles an hour in one temperature zone. Consequently, while a means to enhance airflow through the goggle eye cavity to prevent fogging greatly enhances anti-fogging, it would be especially nice if the actual airflow can be adjusted by changing the outriggers of the goggle.

Also, because goggle users may frequently use their goggles singularly on some occasions, and in combination with a helmet, or cap or other headwear on different occasions, a system whereby the strap, or cord, securing the goggle to the user's head can be easily altered would greatly enhance the utility of a pair of goggles. Such a system would allow the user to easily attach and detach any of a number of means of biased attachment of the goggles to the head of a user such as a strap for encircling the head, or a cord that is dimensioned to engage a mount on a helmet. Further, mechanical helmet mounting means such as pins that engage the helmet could easily be substituted for the strap or cord mount by simply changing the outrigger attached to the goggle.

There is as such, a pressing need for and easily engageable or disengageable outrigger mounting system for goggles which allow the user to easily engage the goggle to any of a plurality of different mounting systems and concurrently allow for the adjustment of the airflow characteristics through the goggle. Such a system should not require any tools and should be simple and easy to operate and engage.

### SUMMARY OF THE INVENTION

The device herein disclosed features a system of rotationally engageable outriggers for goggles which may be worn on the head of the user either with or without a helmet or other headgear. The outriggers are dimensioned to have aerodynamic exterior surfaces that work to maximize air flow through the eye cavity behind the goggle lens and minimize or eliminate fogging of the lens by forming negative air pressure areas adjacent to apertures communicating through the outrigger.

The outriggers would come in a plurality of different configurations to allow for a plurality of different mountings to either the head or a helmet on the head of a user. When the outriggers are adapted for cooperative engagement with a helmet, the helmet goggle combination yields an especially aerodynamic combined helmet and goggle surface, yet still maximizes airflow through the eye cavity behind the lens.

The easy mounting system is accomplished by having the outriggers engaged with a strap, cord, pin, or other means for attachment to the head of a user at a trailing end, and a biased or other hinged engagement to the goggle at the engagement end of the outrigger.

The outriggers being formed of plastic or similar molded material are easily manufactured in a plurality of different shapes to increase or decrease negative pressure adjacent to their apertures. Further, the size and directional flow of the apertures may be varied in different outriggers to both change the volume and direction of the exhaust flowing from the eye cavity during use. The outriggers can be provided in a kit form with many different configured outriggers available to the user and could also be provided in hundreds of different configurations by vendors to individual users depending on the airflow characteristics and type of mounting to their head that they require for their intended use.

An object of this invention is to provide an attachment system for goggles which is aerodynamically dimensioned to maximize airflow from the eye cavity through an outrigger aperture to minimize fogging.

Another object of this invention is the provision of such a goggle mounting system that is easily attached to a goggle that allows the goggle to be configured for wear with or without a helmet.

A further object of this invention is providing such a goggle attachment system which when used in combination with a helmet, still provides increased airflow through the eye cavity of the goggle.

An additional object of this invention is the provision of such a goggle mounting system that provides for mounting apertures available in a wide variety of configurations for mounting to the head of a user either as a kit or by making numerous interchangeable parts available for sale.

Yet an additional object of this invention is the provision of such a cooperatively engaging mounting outrigger that allows the user to adjust both the amount and direction of exhaust through the outrigger aperture from the eye cavity of the goggle.

Further objects of the invention will be brought out in the following part of the specification, wherein a detailed description is for the purpose of fully disclosing the invention without placing limitations thereon.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a side exploded view of the goggle outrigger mounting system showing the outrigger disengaged from the goggle and the compressibly engageable pins on the outrigger.

FIG. 2 is a side view of the device showing a second preferred embodiment of the goggle engageable outrigger which uses a removable pin for a hinged engagement with the goggle.

FIG. 3 depicts the trailing end of the outrigger with a cord type attachment extending therefrom which could be substituted for the strap shown in FIGS. 1 and 2.

FIG. 4 depicts the trailing end of the outrigger with a pin type attachment extending therefrom which could be substituted for the other types of biased engagement when mounting to a helmet with engageable slots.

### DETAILED DESCRIPTION OF THE PREFERRED EMBODIMENT EMBODIMENTS OF THE INVENTION

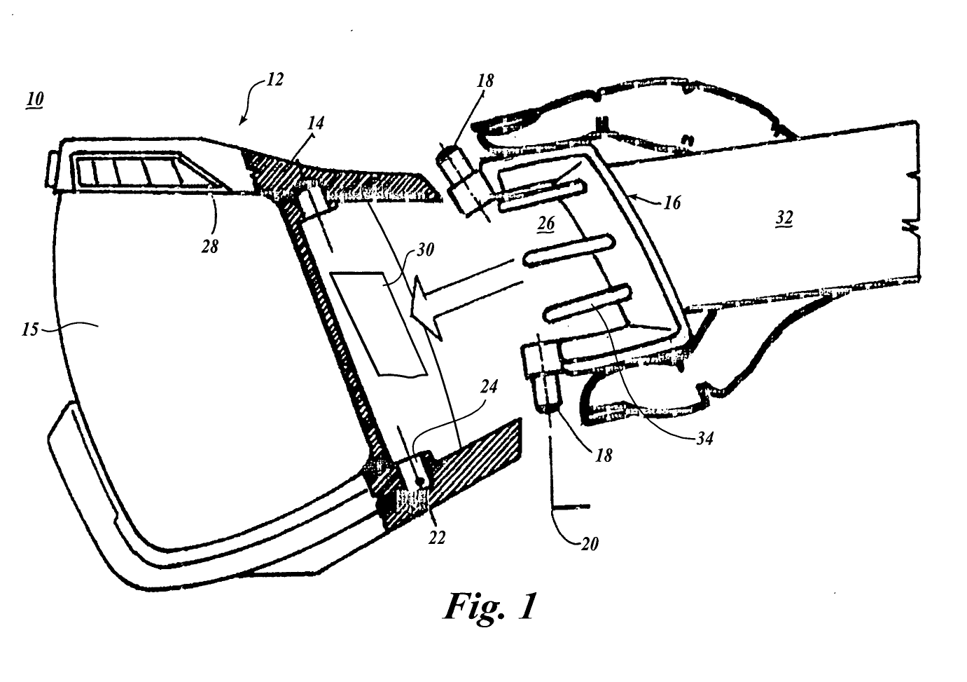

Referring now to the drawings, FIGS. 1-4 depict the various embodiments and engagements of the disclosed outrigger device 10 adapted for engagement with a goggle 12 at the goggle sidewalls 14.

As noted, FIG. 1 is a side exploded view of the goggle outrigger mounting system device 10 showing the outrigger 16 disengaged from its attachment to the sidewall 14 of the goggle 12. In this embodiment, engagement and disengagement is achieved by compressing the top and bottom walls of the outrigger 16 adjacent to the engagement end of the outrigger. The top and bottom walls are by default formed to position two pins 18 in positions where their pin axis 20 is aligned with a cavity axis 22 running between two receiving cavities 24 formed in the goggle sidewall 14. Compressing the top and bottom walls allows the pins 18 to be disengaged from a position where the pin axis 20 and cavity axis 22 are inline with the pins engaged, to a disengaged position where the outrigger 16 may be removed.

As shown, simple pressure on the top and bottom wall of the outrigger 16 with the thumb and fingers, is enough to allow for this engagement and disengagement of the outriggers 16. As such, a kit of outriggers adapted for such a compressible engagement and disengagement are all easily mounted and dismounted.

An aperture 26 communicates through the outrigger 16 and when in the mounted position provides for venting of the eye cavity 28 which communicates immediately adjacent to the aperture 26 through side venting apertures 30 communicating through the sidewalls 14 on either side of the lens 15.

The outriggers are dimensioned to have aerodynamic exterior surfaces that work to maximize airflow through the eye cavity behind the goggle lens and minimize or eliminate fogging of the lens by forming negative air pressure areas adjacent to apertures communicating through the outrigger.

The outriggers 16 may be provided in a plurality of different configurations to allow for a plurality of different mountings to either the head or a helmet on the head of a user. The outriggers 16 or the sidewall 14 in front of the outriggers 16 have exterior surfaces which are dimensioned in an aerodynamic fashion to maximize airflow through the eye cavity 28 behind the goggle lens forming negative air pressure areas adjacent to apertures 26 communicating through the outrigger 16 with the side apertures 30.

The outriggers 16 would come in a plurality of different configurations to allow for a plurality of different mountings to either the head or a helmet on the head of a user. This easy mounting system is accomplished by having the outriggers engaged with a strap 32, cord 34, pin 36, or other means for attachment to the head of a user at a trailing end, and a biased or other hinged engagement to the goggle 12 at the engagement end of the outrigger 16.

The outriggers 16 and sidewalls 14 being formed of plastic or similar molded material are easily manufactured in a plurality of different shapes to increase or decrease negative pressure adjacent to the apertures 26. Further, the size of the aperture 26 may be varied in different outriggers 16 to both change passing air volume. Fins 34 may be employed to alter the direction of the exhaust flowing from the eye cavity 28 during use since different activities might require an upward, downward, or neutral angle of the exhaust for best performance.

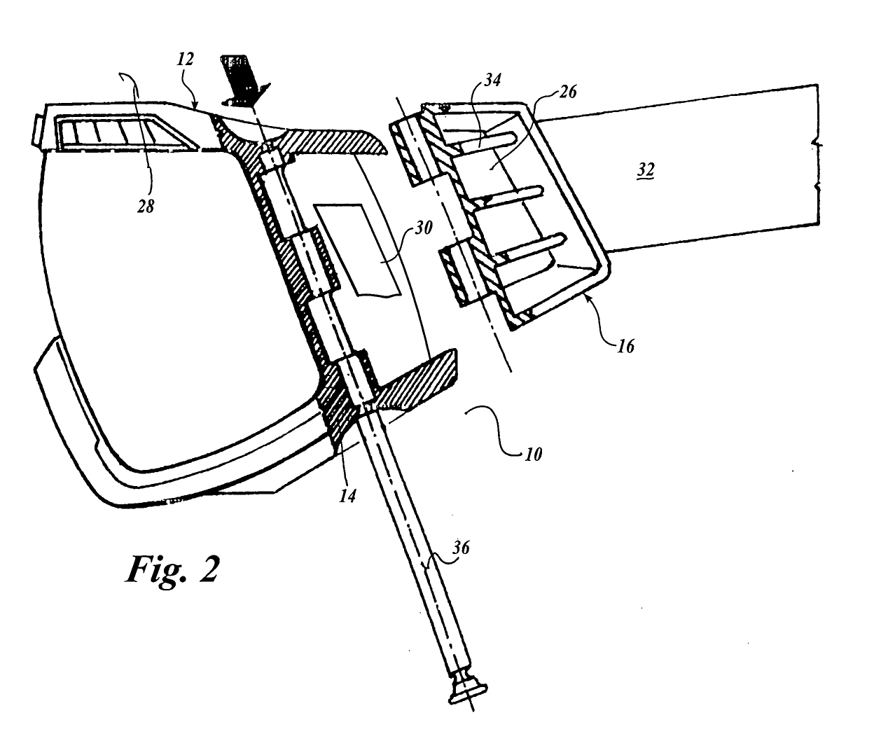

FIG. 2 is a side view of the device showing a second preferred embodiment of the goggle engageable outrigger which uses a removable rod 36 that provides for the hinged engagement with the goggle 12. This engagement like the others, allows the outrigger 16 to rotate toward and away from the goggle 12 during use to adapt for large and small heads and helmets while still yielding the low pressure area adjacent to the aperture 26.

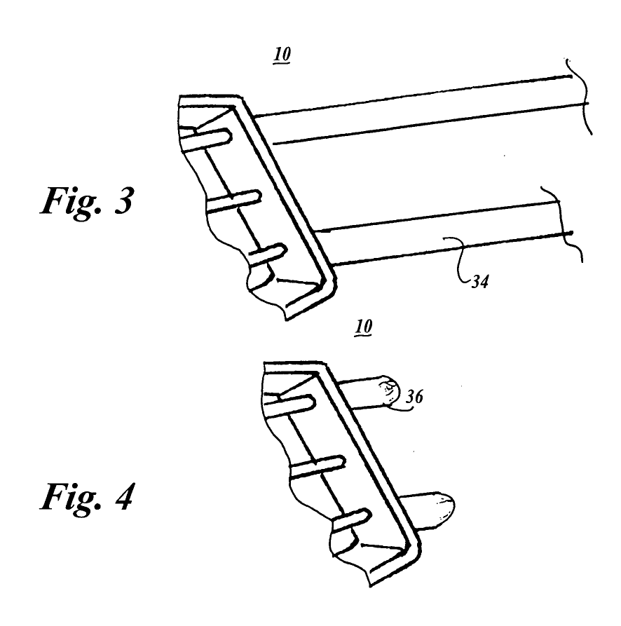

For different mounting situations, the attachment of FIG. 3 can be used interchangeably with those of the other figures at the trailing end of the outrigger 16 with a cord 34 type attachment extending therefrom. For helmet engagement FIG. 4 depicts the trailing end of the outrigger 16 with a pin 36 attachment extending therefrom which could be substituted for the other types of biased engagement when mounting to a helmet with engageable slots.

While all of the fundamental characteristics and features of the present invention have been described herein, with reference to particular embodiments thereof, a latitude of modification, various changes and substitutions are intended in the foregoing disclosure, and it will be apparent that in some instance, some features of the invention will be employed without a corresponding use of other features without departing from the scope of the invention as set forth. It should be understood that such substitutions, modifications, and variations may be made by those skilled in the art without departing from the spirit or scope of the invention. Consequently, all such modifications and variations are included within the scope of the invention.

While the preferred embodiment of the invention has been illustrated and described, it will be appreciated that various changes can be made therein without departing from the spirit and scope of the invention.

1. A goggle engageable mounting system comprising:
an outrigger, said outrigger having an attachment end, a trailing end, and having a venting aperture communicating therethrough;
finger releasable means for detachable engagement of said outriggers to the sidewall of a goggle;
means for attachment of said outrigger to the head of a wearer engaged with said trailing end; and
one or both of said sidewall and said aperture dimensioned on an exterior surface creating a low air pressure area immediately adjacent to said venting aperture when moving air travels over said exterior surface, whereby air flow exhausting from the interior of a goggle to said side aperture is enhanced by the force of said low pressure area acting to draw higher pressure air in said interior of a goggle through said side aperture.

2. The goggle of claim 1, wherein said finger releasable means for engagement of said outriggers to the sidewall of a goggle comprises:
said outrigger having a top side and a bottom side;
said outrigger having pins projecting at said engagement end from said top side and said bottom side; and
said top side finger compressible toward said bottom side whereby said pins may be engaged or disengaged from mating cavities in said sidewall of a goggle.

3. The helmet engageable goggle of claim 1, further comprising fins located in said side aperture to direct airflow therethrough.

4. A sport goggle, comprising:
a goggle body having an exterior surface and an interior surface, said interior surface defining an eye cavity with the face of a wearer, said goggle body having side portions;
an outrigger having a ventilation aperture communicating with the eye cavity;
means for releasably attaching the outrigger to the goggle body to utilize different outrigger configurations with the goggle body; and
wherein one or both of the goggle body side wall portions and outrigger apertures are shaped and dimensioned to form an exterior surface that creates a low air pressure area immediately adjacent the ventilation aperture of the outrigger when moving air flows over the exterior surface of the goggle body and the outrigger, whereby air flow exhausting from the eye cavity through said outrigger aperture is enhanced by the force of the low pressure area acting to draw high pressure air from said eye cavity through said outrigger aperture.

5. A goggle according to claim 4, further comprising fins positioned in the outrigger venting aperture to direct air flow therethrough.

6. The goggle according to claim 4, wherein said releasable attaching means, comprising:
said outrigger having a top side and a bottom side;
said outrigger having pins projecting from said top and bottom sides of the outrigger, said pins positioned and sized to be receivable within correspondingly shaped apertures formed in the goggled body; and
wherein said outrigger top and bottom sides being compressible, whereby said pins may be engaged into or disengaged from said mating apertures.
